(19) United States
(12) Patent Application Publication
(10) Pub. No.: US 2024/0233858 A1
(43) Pub. Date: Jul. 11, 2024

(54) MICROORGANISM INFLUENCED CORROSION PREDICTION

(71) Applicant: Saudi Arabian Oil Company, Dhahran (SA)

(72) Inventors: Budoor Ali Nasser, Thuwal (SA); Christian M. Canto Maya, Dhahran (SA)

(21) Appl. No.: 18/153,076

(22) Filed: Jan. 11, 2023

(51) Int. Cl. G16B 5/00; C12Q 1/02; G16B 40/00; G16B 45/00

(52) U.S. Cl. CPC G16B 5/00; C12Q 1/025; G16B 40/00; G16B 45/00

(57) ABSTRACT

A method can be performed by a computer system, the method can include inputting into a microbiologically influenced corrosion (MIC) prediction model one or more operating conditions for a physical asset in a production field; inputting into the MIC prediction model a plurality of biomarkers, each of the plurality of biomarkers uniquely identifying one species of microorganism from a plurality of species of microorganisms; determining, from the MIC prediction model, a likelihood of growth of one or more species of microorganisms from the plurality of species of microorganisms based on the one or more operating conditions for the physical asset and the plurality of biomarkers; and determining, from the likelihood of the growth of one or more species of microorganisms, a mitigation strategy for preventing growth of each of the one or more species of microorganisms within the physical asset.

### TECHNICAL FIELD

The present disclosure pertains to the use of gene maps to predict the likelihood of bio-deterioration in production assets by microbiologically influenced corrosion.

### BACKGROUND

Corrosion in pipelines and other production assets can result in damage to production sites and can cause substantial economic losses. Biological microbial activities can cause microbiologically influenced corrosion (MIC) and bio-deterioration.

### SUMMARY

The present disclosure describes techniques that can be used for predicting the likelihood of MIC-based corrosion in a production asset using gene mapping and operating conditions.

In some implementations, a computer-implemented method includes the following.

Aspects of the embodiments are directed to a computer-implemented method that includes inputting into a microbiologically influenced corrosion (MIC) prediction model one or more operating conditions for a physical asset in a production field; inputting into the MIC prediction model a plurality of biomarkers, each of the plurality of biomarkers uniquely identifying one species of microorganism from a plurality of species of microorganisms; determining, from the MIC prediction model, a likelihood of growth of one or more species of microorganisms from the plurality of species of microorganisms based on the one or more operating conditions for the physical asset and the plurality of biomarkers; and determining, from the likelihood of the growth of one or more species of microorganisms, a mitigation strategy for preventing growth of each of the one or more species of microorganisms within the physical asset.

In some embodiments, each of the plurality of biomarkers comprises a 16S ribosomal ribonucleic acid (rRNA) gene for a corresponding microorganism species.

In some embodiments, the MIC prediction model uses a 16S rRNA gene for the corresponding microorganism species to correlate a geographic region where the production field is located with a likelihood of microorganism growth within the physical asset in view of the operating conditions.

In some embodiments, determining the likelihood of growth of one or more species of microorganisms includes predicting a concentration of each species of microorganism in parts per million (PPM).

Some embodiments can include determining whether the predicted concentration of each species of microorganism meets or exceeds a threshold value, the threshold value establishing a minimum microorganism concentration value; and determining the mitigation strategy for each microorganism based on the predicted concentration of the microorganism meeting or exceeding the threshold value.

Some embodiments can include, if the predicted concentration of a microorganism meets or exceeds the threshold value, inputting the predicted concentration of each of the species of microorganism into a mechanistic model; and predicting one or both of 1) a likelihood of corrosion in the physical asset due to the predicted concentration of each species of microorganism and 2) a rate of corrosion in the physical asset due to the predicted concentration of each species of microorganism.

Some embodiments can include, for each of the microorganisms having a predicted concentration that meets or exceeds the threshold value, determining a metabolic pathway for each of the species of microorganisms; and determining the mitigation strategy for each of the species of microorganisms based, at least in part, on the metabolic pathway.

In some embodiments, the mitigation strategy includes one or both of a) modifying the operating conditions to slow or prevent to the growth of one or more species of microorganisms; or b) adding a biocide treatment for the one or more species of microorganisms to the operating conditions.

Some embodiments can include generating a database of biomarkers for the production asset based, at least in part, on the operating conditions associated with operation of the production asset and the determined likelihood of a growth of one or more species of microorganisms in the production asset.

Some embodiments can include testing the physical asset for MIC; determining a concentration of one or more species of microorganisms; determining an identity of the one or more species of microorganisms based on a genetic biomarker of the identified microorganism; comparing the concentration of the one or more species of microorganisms with the likelihood of growth of one or more species of microorganisms predicted by the MIC prediction model; and updating the MIC prediction model based on the comparison.

Aspects of the embodiments are directed to one or more non-transitory, computer-readable storage media storing one or more instructions executable by a computer system to perform operations including inputting into a microbiologically influenced corrosion (MIC) prediction model one or more operating conditions for a physical asset in a production field; inputting into the MIC prediction model a plurality of biomarkers, each of the plurality of biomarkers being unique to a species of microorganism; determining, from the MIC prediction model, a likelihood of a growth of one or more species of microorganisms based on the operating conditions for the physical asset and the plurality of biomarkers; and determining, from the likelihood of the growth of one or more species of microorganisms, a mitigation strategy for preventing growth of the one or more species of microorganisms within the physical asset.

In some embodiments, each of the plurality of biomarkers includes a 16S ribosomal ribonucleic acid (rRNA) gene for a corresponding microorganism species.

In some embodiments, the MIC prediction model uses a 16S rRNA gene for the corresponding microorganism species to correlate a geographic region where the production field is located with a likelihood of microorganism growth within the physical asset in view of the operating conditions.

In some embodiments, determining the likelihood of growth of one or more species of microorganisms includes predicting a concentration of microorganism in parts per million (PPM).

Some embodiments can include determining whether the predicted concentration of a microorganism meets or exceeds a threshold value, the threshold value establishing a minimum microorganism concentration value; and determining a mitigation strategy for the microorganism based on the predicted concentration of the microorganism meeting or exceeding the threshold value.

Some embodiments can include, if the predicted concentration of a microorganism meets or exceeds the threshold value inputting the predicted concentration of the microorganism into a mechanistic model; and predicting one or both of 1) a likelihood of corrosion in the physical asset due to the predicted concentration of the microorganism and 2) a rate of corrosion in the physical asset due to the predicted concentration of the microorganism.

Some embodiments can include, for each of the microorganisms having a predicted concentration that meets or exceeds the threshold value, determining a metabolic pathway for each of the microorganisms; and determining a mitigation strategy for each of the microorganisms based, at least in part, on the metabolic pathway.

In some embodiments, the mitigation strategy includes one or both of a) modifying the operating conditions to slow or prevent to the growth of one or more species of microorganisms; or b) adding a biocide treatment for the one or more species of microorganisms to the operating conditions.

Some embodiments can include generating a database of biomarkers for the production asset based, at least in part, on the operating conditions associated with operation of the production asset and the determined likelihood of a growth of one or more species of microorganisms in the production asset.

Aspects of the embodiments are directed to a computer-implemented system that can include one or more processors; and a non-transitory computer-readable storage medium coupled to the one or more processors and storing programming instructions for execution by the one or more processors, the programming instructions instructing the one or more processors to perform operations including inputting into a microbiologically influenced corrosion (MIC) prediction model one or more operating conditions for a physical asset in a production field; inputting into the MIC prediction model a plurality of biomarkers, each of the plurality of biomarkers being unique to a species of microorganism; determining, from the MIC prediction model, a likelihood of a growth of one or more species of microorganisms based on the operating conditions for the physical asset and the plurality of biomarkers; and determining, from the likelihood of the growth of one or more species of microorganisms, a mitigation strategy for preventing growth of the one or more species of microorganisms within the physical asset.

The previously described implementation is implementable using a computer-implemented method; a non-transitory, computer-readable medium storing computer-readable instructions to perform the computer-implemented method; and a computer-implemented system including a computer memory interoperably coupled with a hardware processor configured to perform the computer-implemented method/the instructions stored on the non-transitory, computer-readable medium.

The subject matter described in this specification can be implemented in particular implementations, so as to realize one or more of the following advantages. For example, aspects of the present disclosure facilitate the identification of microbes that could proliferate within a pipeline or other oil field asset and the identification of a mitigation strategy to avoid MIC-based bio-deterioration. A computer system can then cause the mitigation strategy to be implemented.

The details of one or more implementations of the subject matter of this specification are set forth in the Detailed Description, the accompanying drawings, and the claims. Other features, aspects, and advantages of the subject matter will become apparent from the Detailed Description, the claims, and the accompanying drawings.

### BRIEF DESCRIPTION OF THE DRAWINGS

FIG. 1A is a schematic diagram of an example computational system that includes a microorganism influenced corrosion prediction model fed by a gene map and operating conditions in accordance with embodiments of the present disclosure.

FIG. 1B is a schematic diagram of an example training model for the microorganism influenced corrosion prediction model in accordance with embodiments of the present disclosure.

FIG. 2 is a process flow diagram for predicting the growth of one or more microorganisms within a physical asset using a microorganism influenced corrosion prediction model fed by a gene map and operating conditions in accordance with embodiments of the present disclosure.

FIG. 3 is a block diagram illustrating an example computer system used to provide computational functionalities associated with described algorithms, methods, functions, processes, flows, and procedures as described in the present disclosure, according to some implementations of the present disclosure.

Like reference numbers and designations in the various drawings indicate like elements.

### DETAILED DESCRIPTION

The following detailed description describes techniques for predicting the growth of one or more microorganisms within a physical asset, such as a pipeline, operating in a production field using a prediction model that relies on operating conditions for the physical asset and a gene map. Various modifications, alterations, and permutations of the disclosed implementations can be made and will be readily apparent to those of ordinary skill in the art, and the general principles defined may be applied to other implementations and applications, without departing from scope of the disclosure. In some instances, details unnecessary to obtain an understanding of the described subject matter may be omitted so as to not obscure one or more described implementations with unnecessary detail and inasmuch as such details are within the skill of one of ordinary skill in the art. The present disclosure is not intended to be limited to the described or illustrated implementations, but to be accorded the widest scope consistent with the described principles and features.

this disclosure describes systems and processes to predict whether and to what extent microorganisms could proliferate within a physical asset, such as a pipeline, using a bio-defect model fed by a database of biological markers (“biomarkers”) from a gene map. The gene map can provide biomarkers to correlate operating conditions within a pipeline that promote the proliferation of microorganisms. These microbial species could be used as a preliminary risk ranking for the bio-defects, which can include bio-corrosion and bio-degradation of metals or other materials within the physical asset. The gene map will provide information pertaining to how the microorganism is responsible for the development of pipeline bio-defects, which can be used in a mechanistic model to understand the corrosion pathways caused by the predicted concentration of each microorganism that could form within the physical asset.

The gene map will support the development of the MIC prediction method in the bio-defects assessment in oil and gas assets, such as pipelines. This prediction method will support decision making for operating conditions, mitigation treatments, and other aspects of production, especially in remote areas and subterranean assets, where inspection and testing is difficult or not possible. The MIC prediction method described herein is expected to help in the reduction of preventive costs by mitigating microorganism influenced corrosion and degradation. The integration of the gene map into a predictive method would help to develop new methods to suppress bio-defects by choosing appropriate mitigation strategies.

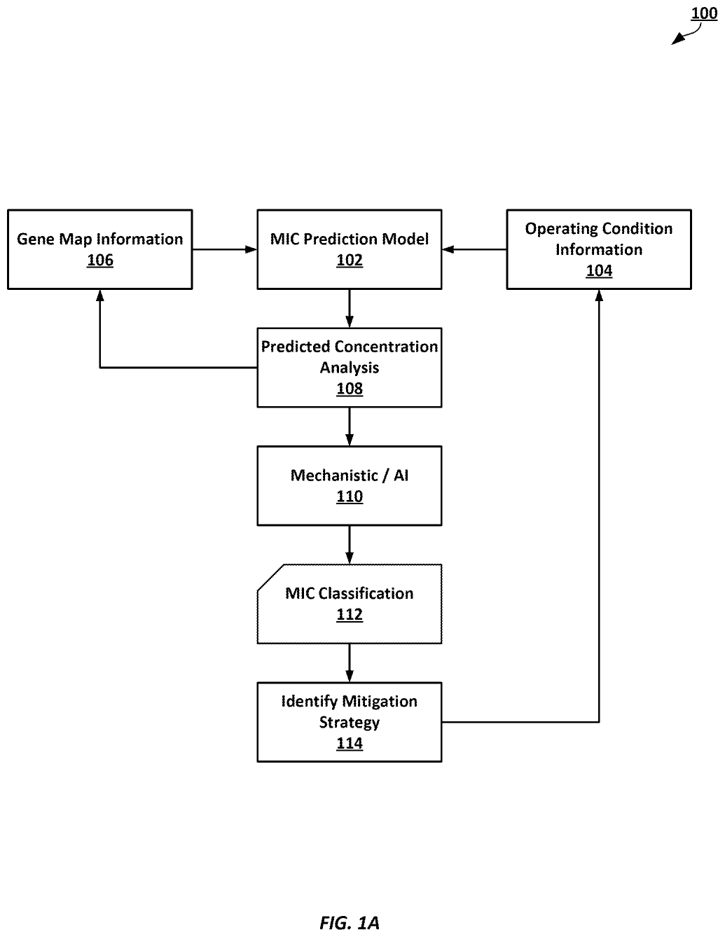

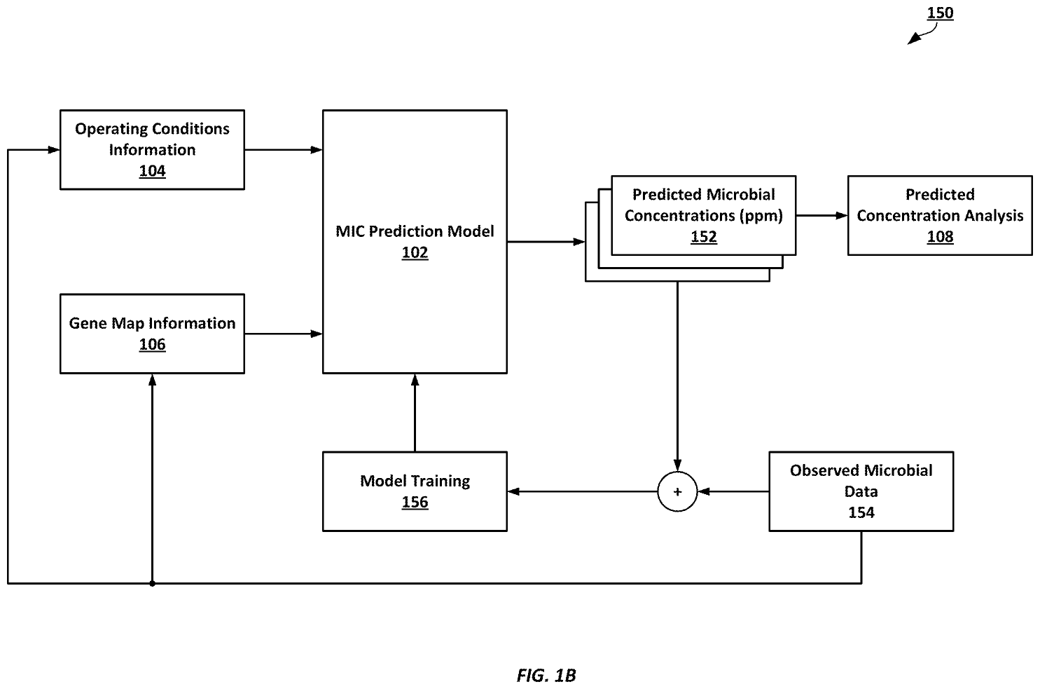

FIG. 1A is a schematic diagram of an example computational system 100 that includes a microorganism influenced corrosion (MIC) prediction model fed by a gene map and operating conditions in accordance with embodiments of the present disclosure. The computational system 100 includes an MIC prediction model 102. The MIC prediction model (also referred to as a bio-defects model) can make use of machine learning, deep learning, or other type of artificial intelligence, to predict whether and to what extent one or more microorganism species can grow within a physical asset based on operating conditions for the physical asset. This MIC prediction model 102 tries to help to identify detrimental bacteria by field and the likelihood of its proliferation depending on the conditions along the pipelines. Several tabular classification methods such as XGboost, CATboost, LightGBM, and TabPFN can be used to develop the MIC prediction model 102.

The MIC prediction model 102 can make use of machine learning, such as cluster techniques and environmental predictions. The machine learning models can include neural networks that use supervised, unsupervised, or a hybrid supervised training process. For example, the MIC prediction model 102 can rely on a supervised method to help identify the type of microbial growth under specific operating conditions. Once the microbial growth can be identified and classified, another mechanistic model can be used predict the metabolic pathway for one or more microorganisms that would affect the physical asset if allowed to proliferate.

The MIC prediction model 102 includes as an input operating conditions information 104 as a way to relate MIC damage with operating conditions. Operation conditions information 104 that can influence microbial corrosion in pipelines includes temperature, pH, carbon dioxide (CO2) and hydrogen sulfide (H2S) content, water chemistry, flow velocity, microbial contamination, oil or water wetting, and composition and surface conditions of the metal. Operating conditions that are known to affect MIC in particular, include flow rate (velocity), temperature, pressure, pH, oxygen level, cleanliness of the system, and water quality and level. Other operating conditions information can also be used and are within the scope of this disclosure, such as the materials used in the physical asset (e.g., type of metal or composite material).

The MIC prediction model 102 also includes as an input gene map information 106. The gene map information 106 can include a unique biological marker (biomarker) for one or more species of microorganisms. Gene map information 106 can be used to identify a specific microorganism that could contribute to the formation of defects (such biocorrosion and biodegradation) in the pipeline or other physical asset. The gene map information 106 can include a unique biomarker to identify favorable condition that promotes the bacteria proliferation. Generally, the biomarker can be a diagnostic biomarker (e.g., those used for early detection) to screening the field. In crude oil implementations, the biomarker can be a deoxyribose nucleic acid (DNA)-based biomarker because for crude oil, it is difficult to use protein-, lipid-, or sugar-based biomarkers. For example, a 16S ribosomal ribonucleic acid (rRNA) gene can be used as a biomarker to uniquely identify a species of bacterium.

In addition, the biomarker can identify or be associated with a set of operating conditions under which the species of microorganism can flourish. Consequently, gene map information 106 being integrated with the capabilities of the MIC prediction model 102 can help to predict the proliferation of specific microorganisms, help to identify specific mitigation strategies to combat the growth of such microorganisms in view of metabolic pathways, and reduce preventive cost against biocorrosion and save spending on maintenance.

Gene map information 106 is used as a database of biomarkers to improve the correlation between the operating conditions information 104 and the prediction of microorganism proliferation by the MIC prediction model 102. One advantage of the MIC prediction model 102 described herein is that the use of the gene map information 106 in the MIC prediction model 102 can facilitate the prediction of how microbial activity changes depending on the geographic region in which the physical asset is operating. Understanding the specific microbial structure can help to identify where the MIC can occur along the pipelines depending on the nutrients and environmental distribution.

For each production system, a unique MIC prediction model 102 can be developed. Because each production system can operate in a different geographic area, each with its own set of operating conditions and potential set of microbial presence, each MIC prediction model 102 can be updated with a decreasing subset of possible microorganisms that are predicted to grow and are subsequently found within the physical asset. The gene map information 106, thus, can be tuned for each production system using both the output of the MIC prediction model 102 and real-world data (e.g., validation data).

FIG. 1B is a schematic diagram of an example training model 150 for the microorganism influenced corrosion prediction model 100 in accordance with embodiments of the present disclosure. As described before, the MIC prediction model 102 can include as inputs operation conditions information 104 and gene map information 106. The output of the MIC prediction model 102 can include predictions of concentrations (e.g., in parts per million) of one or more species of microorganisms (generally, predicted microbial concentrations 152). The predicted microbial concentration(s) 152 can be compared against observed microbial data 154. For example, for the specific production system in a geographic area, analyses can be performed to identify and quantify microbes in production fluids, on physical assets, or elsewhere. Model training 156 can use the comparison to update weights for the MIC prediction model 102 or other hidden layer values. In addition, observed microbial data 154 can be used to update the gene map information 106 and the operation conditions information 104.

Returning to FIG. 1A, the MIC prediction model 102 can output a predicted concentration of microbes in water, in parts per million (ppm) or other metric. The predicted concentration can be analyzed by a predicted concentration analysis algorithm 108. The predicted concentration analysis algorithm 108 can compare the predicted concentration from the MIC predication model 102 with a threshold concentration value. The threshold concentration value can define a minimum concentration below which the predicted concentration of corresponding species of microorganism would be undetectable or would be insufficiently high to result in MIC effects. A threshold concentration can be established for each species of microorganism. In some embodiments, predicted concentrations below 1 ppm are considered below detectable limits. Such a threshold can be used to predict whether an identified mitigation strategy might be sufficient to reduce microbial populations to sufficiently small quantities to prevent MIC.

When the MIC prediction model 102 outputs a predicted microbial concentration at or above the threshold concentration value, the concentration values can be input into the mechanistic model 110. The mechanistic model 110 can be used to computationally determine physical, biological, chemical, or other types of metabolic pathways for MIC based on the predicted concentrations of microorganisms from the MIC prediction model 102. The mechanistic model 110 can identify, therefore, whether and how the one or more species of microorganisms could cause biodefects, biocorrosion, or biodegradation of physical assets in each production field.

Predicting one or more identified species of microorganism using the gene map information 106 would help to classify the type of MIC that could take place. For example, the specific metabolic pathways that occur to cause the bio-corrosions & biodegradation by the proliferation of the one or more species of microorganism. This classification can improve the development of new methods to suppress bio-corrosions & biodegradation by appropriate selection of biocides or antibiotics. Such a classification can be done through mechanistic model 110 which yields MIC classifications 112. The MIC classifications 112 can then be used to determine one or more mitigation strategies by a mitigation strategies algorithm 114 for mitigation or preventing the growth or proliferation of bio-defect-causing microorganisms.

The mitigation strategy (or strategies) for the one or more species of microorganisms can be used to update the operating conditions 104. As mentioned before, knowledge about what type(s) of microorganisms are likely to grow in the specific environment of the production field can be used to choose the right biocide or antibiotic to prevent or mitigate such proliferation. For example, a field's operating conditions may include the use of a first biocide in process fluid in a physical asset. The MIC prediction model 102 could reveal that a dominant bacteria that is likely to grow in that physical asset can break down or withstand that first biocide (or even use the biocide as a nutrient). The mitigation strategy could include using an additional biocide with a choke dose. In addition, or in the alternative, a mitigation strategy could include changing the operating conditions to address microbial growth without unduly affecting production. The operation condition controls the bacteria growth and causes the MIC; and it may be possible to change the operating conditions to address the risk of microbial growth by tuning the operating conditions. After the mitigation strategies are determined, each mitigation strategy can then be implemented. In scenarios where multiple mitigations strategies are recommend, all mitigation strategies can be implemented, or a subset can be implemented to iteratively check the efficacy of the recommended strategy. The mitigations strategies can be implemented automatically (e.g., by automatically changing operating conditions) by a computer system. Such a computer system can be the same system that performs the MIC prediction, or can be a different system that receives the recommendation but controls one or more aspects of the production field. To the extent that biocides, antibiotics, or other additives are to be included in the operating conditions, the computer system can control the inclusion of such additives or can update operating conditions instructions or recipes to cause a human operator to include the additives.

The predicted concentration output from the MIC prediction model 102 can also be used to update database of the gene map information 106. The update can remove entries from the database that are unlikely to be found in the specific production field. In addition, microorganisms with higher predicted concentrations in that production field can be ranked higher. Species of microorganisms with predicted concentrations below This way, the gene map information 106 can uniquely represent the operating conditions and environmental conditions associated with the specific production field or even with specific physical assets with each production field. The granularity of the gene map information 106 can be implementation specific.

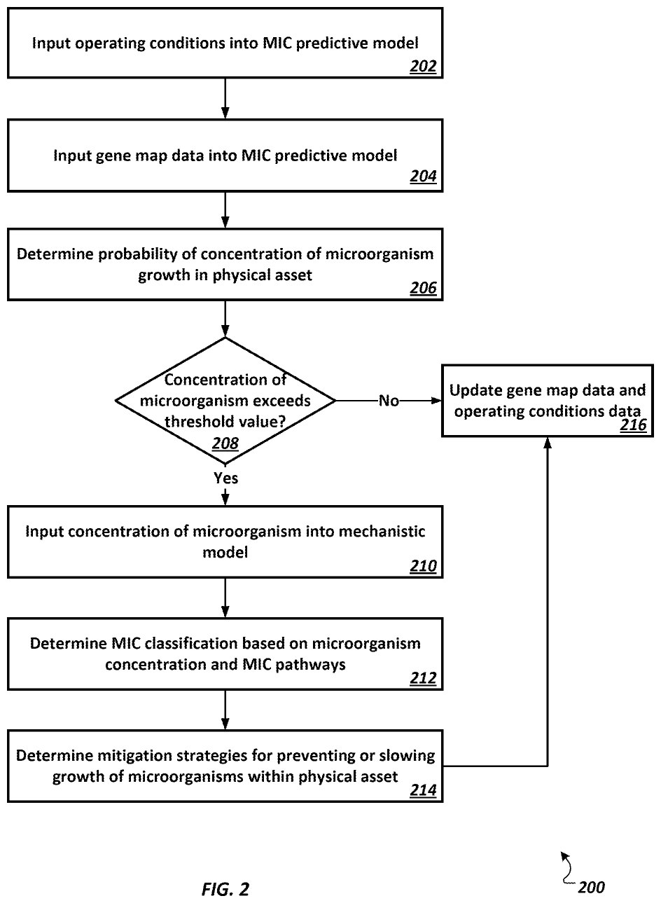

FIG. 2 is a process flow diagram 20 for predicting the growth of one or more microorganisms within a physical asset using a microorganism influenced corrosion prediction model fed by a gene map and operating conditions in accordance with embodiments of the present disclosure.

For clarity of presentation, the description that follows generally describes method 200 in the context of the other figures in this description. However, it will be understood that method 200 can be performed, for example, by any suitable system, environment, software, and hardware, or a combination of systems, environments, software, and hardware, as appropriate. In some implementations, various steps of method 200 can be run in parallel, in combination, in loops, or in any order.

At 202, operating conditions information is input into an MIC prediction model. At 204, gene map data is input into an MIC prediction model. Steps 202 and 204 can occur concurrently (or without regard to order, since they are both inputs into the MIC prediction model and both are used by the MIC prediction model).

From 204, method 200 proceeds to 206.

At 206, the MIC prediction model outputs a prediction of the concentration of one or more species of microorganisms based on the operating conditions information and the gene map information.

From 206, method 200 proceeds to 208.

At 208, the predicted concentrations of each of the identified species of microorganisms is compared against a threshold concentration value. The threshold concentration value can establish a floor below which the predicted concentration is too low to be detected. For predicted concentrations that fall below the threshold, the gene map data and the operating conditions can be updated to reflect the fact that for at least one identified species of microorganism, the concentration levels are too low to be detected. This can be because the operating conditions do not favor the growth of the microbe, or the mitigation strategies already in place have address the specific microbe in a satisfactory way.

From 208, method 200 proceeds to 210. At 210, for concentrations of each species of microorganism that meet or exceed the threshold concentration value, the predicted concentrations of each species of microorganism can be input into a mechanistic model. At 212, the mechanistic model can classify the biodefect by computationally determining the metabolic pathways of degradation for by the predicted concentration of each species of microorganism.

At 214, one or more mitigation strategies can be formulated based on the MIC classifications identified by the mechanistic model. The mitigation strategies can then be used to update the operation conditions and the gene map data.

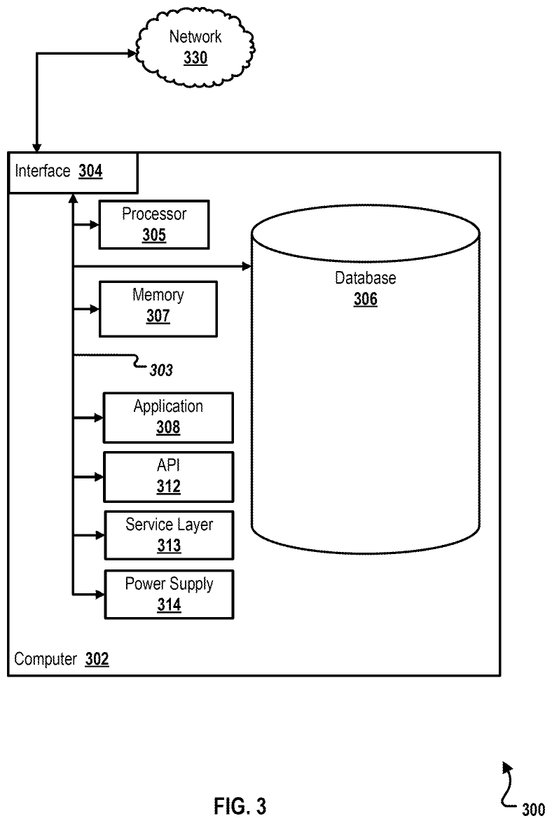

FIG. 3 is a block diagram of an example computer system 300 used to provide computational functionalities associated with described algorithms, methods, functions, processes, flows, and procedures described in the present disclosure, according to some implementations of the present disclosure. The illustrated computer 302 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 302 can include input devices such as keypads, keyboards, and touch screens that can accept user information. Also, the computer 302 can include output devices that can convey information associated with the operation of the computer 302. The information can include digital data, visual data, audio information, or a combination of information. The information can be presented in a graphical user interface (UI) (or GUI).

The computer 302 can serve in a role as a client, a network component, a server, a database, a persistency, or components of a computer system for performing the subject matter described in the present disclosure. The illustrated computer 302 is communicably coupled with a network 330. In some implementations, one or more components of the computer 302 can be configured to operate within different environments, including cloud-computing-based environments, local environments, global environments, and combinations of environments.

At a top level, the computer 302 is an electronic computing device operable to receive, transmit, process, store, and manage data and information associated with the described subject matter. According to some implementations, the computer 302 can also include, or be communicably coupled with, an application server, an email server, a web server, a caching server, a streaming data server, or a combination of servers.

The computer 302 can receive requests over network 330 from a client application (for example, executing on another computer 302). The computer 302 can respond to the received requests by processing the received requests using software applications. Requests can also be sent to the computer 302 from internal users (for example, from a command console), external (or third) parties, automated applications, entities, individuals, systems, and computers.

Each of the components of the computer 302 can communicate using a system bus 303. In some implementations, any or all of the components of the computer 302, including hardware or software components, can interface with each other or the interface 304 (or a combination of both) over the system bus 303. Interfaces can use an application programming interface (API) 312, a service layer 313, or a combination of the API 312 and service layer 313. The API 312 can include specifications for routines, data structures, and object classes. The API 312 can be either computer-language independent or dependent. The API 312 can refer to a complete interface, a single function, or a set of APIs.

The service layer 313 can provide software services to the computer 302 and other components (whether illustrated or not) that are communicably coupled to the computer 302. The functionality of the computer 302 can be accessible for all service consumers using this service layer. Software services, such as those provided by the service layer 313, can provide reusable, defined functionalities through a defined interface. For example, the interface can be software written in JAVA, C++, or a language providing data in extensible markup language (XML) format. While illustrated as an integrated component of the computer 302, in alternative implementations, the API 312 or the service layer 313 can be stand-alone components in relation to other components of the computer 302 and other components communicably coupled to the computer 302. Moreover, any or all parts of the API 312 or the service layer 313 can be implemented as child or sub-modules of another software module, enterprise application, or hardware module without departing from the scope of the present disclosure.

The computer 302 includes an interface 304. Although illustrated as a single interface 304 in FIG. 3, two or more interfaces 304 can be used according to particular needs, desires, or particular implementations of the computer 302 and the described functionality. The interface 304 can be used by the computer 302 for communicating with other systems that are connected to the network 330 (whether illustrated or not) in a distributed environment. Generally, the interface 304 can include, or be implemented using, logic encoded in software or hardware (or a combination of software and hardware) operable to communicate with the network 330. More specifically, the interface 304 can include software supporting one or more communication protocols associated with communications. As such, the network 330 or the interface's hardware can be operable to communicate physical signals within and outside of the illustrated computer 302.

The computer 302 includes a processor 305. Although illustrated as a single processor 305 in FIG. 3, two or more processors 305 can be used according to particular needs, desires, or particular implementations of the computer 302 and the described functionality. Generally, the processor 305 can execute instructions and can manipulate data to perform the operations of the computer 302, including operations using algorithms, methods, functions, processes, flows, and procedures as described in the present disclosure.

The computer 302 also includes a database 306 that can hold data for the computer 302 and other components connected to the network 330 (whether illustrated or not). For example, database 306 can be an in-memory, conventional, or a database storing data consistent with the present disclosure. In some implementations, database 306 can be a combination of two or more different database types (for example, hybrid in-memory and conventional databases) according to particular needs, desires, or particular implementations of the computer 302 and the described functionality. Although illustrated as a single database 306 in FIG. 3, two or more databases (of the same, different, or combination of types) can be used according to particular needs, desires, or particular implementations of the computer 302 and the described functionality. While database 306 is illustrated as an internal component of the computer 302, in alternative implementations, database 306 can be external to the computer 302.

The computer 302 also includes a memory 307 that can hold data for the computer 302 or a combination of components connected to the network 330 (whether illustrated or not). Memory 307 can store any data consistent with the present disclosure. In some implementations, memory 307 can be a combination of two or more different types of memory (for example, a combination of semiconductor and magnetic storage) according to particular needs, desires, or particular implementations of the computer 302 and the described functionality. Although illustrated as a single memory 307 in FIG. 3, two or more memories 307 (of the same, different, or combination of types) can be used according to particular needs, desires, or particular implementations of the computer 302 and the described functionality. While memory 307 is illustrated as an internal component of the computer 302, in alternative implementations, memory 307 can be external to the computer 302.

The application 308 can be an algorithmic software engine providing functionality according to particular needs, desires, or particular implementations of the computer 302 and the described functionality. For example, application 308 can serve as one or more components, modules, or applications. Further, although illustrated as a single application 308, the application 308 can be implemented as multiple applications 308 on the computer 302. In addition, although illustrated as internal to the computer 302, in alternative implementations, the application 308 can be external to the computer 302.

The computer 302 can also include a power supply 314. The power supply 314 can include a rechargeable or non-rechargeable battery that can be configured to be either user- or non-user-replaceable. In some implementations, the power supply 314 can include power-conversion and management circuits, including recharging, standby, and power management functionalities. In some implementations, the power-supply 314 can include a power plug to allow the computer 302 to be plugged into a wall socket or a power source to, for example, power the computer 302 or recharge a rechargeable battery.

There can be any number of computers 302 associated with, or external to, a computer system containing computer 302, with each computer 302 communicating over network 330. Further, the terms “client,” “user,” and other appropriate terminology can be used interchangeably, as appropriate, without departing from the scope of the present disclosure. Moreover, the present disclosure contemplates that many users can use one computer 302 and one user can use multiple computers 302.

Described implementations of the subject matter can include one or more features, alone or in combination.

Example 1 is a computer-implemented method including inputting into a microbiologically influenced corrosion (MIC) prediction model one or more operating conditions for a physical asset in a production field; inputting into the MIC prediction model a plurality of biomarkers, each of the plurality of biomarkers uniquely identifying one species of microorganism from a plurality of species of microorganisms; determining, from the MIC prediction model, a likelihood of growth of one or more species of microorganisms from the plurality of species of microorganisms based on the one or more operating conditions for the physical asset and the plurality of biomarkers; and determining, from the likelihood of the growth of one or more species of microorganisms, a mitigation strategy for preventing growth of each of the one or more species of microorganisms within the physical asset.

Example 2 may include the subject matter of example 1, wherein each of the plurality of biomarkers includes a 16S ribosomal ribonucleic acid (rRNA) gene for a corresponding microorganism species.

Example 3 may include the subject matter of example 2, wherein the MIC prediction model uses a 16S rRNA gene for the corresponding microorganism species to correlate a geographic region where the production field is located with a likelihood of microorganism growth within the physical asset in view of the operating conditions.

Example 4 may include the subject matter of any of examples 1-3, wherein determining the likelihood of growth of one or more species of microorganisms includes predicting a concentration of each species of microorganism in parts per million (PPM).

Example 5 may include the subject matter of example 4, and can also include determining whether the predicted concentration of each species of microorganism meets or exceeds a threshold value, the threshold value establishing a minimum microorganism concentration value; and determining the mitigation strategy for each microorganism based on the predicted concentration of the microorganism meeting or exceeding the threshold value.

Example 6 may include the subject matter of example 5, and can also include, if the predicted concentration of a microorganism meets or exceeds the threshold value inputting the predicted concentration of each of the species of microorganism into a mechanistic model; and predicting one or both of 1) a likelihood of corrosion in the physical asset due to the predicted concentration of each species of microorganism and 2) a rate of corrosion in the physical asset due to the predicted concentration of each species of microorganism.

Example 7 may include the subject matter of example 5, and can also include for each of the microorganisms having a predicted concentration that meets or exceeds the threshold value, determining a metabolic pathway for each of the species of microorganisms; and determining the mitigation strategy for each of the species of microorganisms based, at least in part, on the metabolic pathway.

Example 8 may include the subject matter of any of examples 1-7, wherein the mitigation strategy comprises one or both of a) modifying the operating conditions to slow or prevent to the growth of one or more species of microorganisms; or b) adding a biocide treatment for the one or more species of microorganisms to the operating conditions.

Example 9 may include the subject matter of any of examples 1-8, and can also include generating a database of biomarkers for the production asset based, at least in part, on the operating conditions associated with operation of the production asset and the determined likelihood of a growth of one or more species of microorganisms in the production asset.

Example 10 may include the subject matter of any of examples 1-9, and can also include testing the physical asset for MIC; determining a concentration of one or more species of microorganisms; determining an identity of the one or more species of microorganisms based on a genetic biomarker of the identified microorganism; comparing the concentration of the one or more species of microorganisms with the likelihood of growth of one or more species of microorganisms predicted by the MIC prediction model; and updating the MIC prediction model based on the comparison.

Example 11 is a non-transitory, computer-readable storage medium storing one or more instructions executable by a computer system to perform operations comprising inputting into a microbiologically influenced corrosion (MIC) prediction model one or more operating conditions for a physical asset in a production field; inputting into the MIC prediction model a plurality of biomarkers, each of the plurality of biomarkers being unique to a species of microorganism; determining, from the MIC prediction model, a likelihood of a growth of one or more species of microorganisms based on the operating conditions for the physical asset and the plurality of biomarkers; and determining, from the likelihood of the growth of one or more species of microorganisms, a mitigation strategy for preventing growth of the one or more species of microorganisms within the physical asset.

Example 12 may include the subject matter of example 11, wherein each of the plurality of biomarkers comprises a 16S ribosomal ribonucleic acid (rRNA) gene for a corresponding microorganism species.

Example 13 may include the subject matter of example 12, wherein the MIC prediction model uses a 16S rRNA gene for the corresponding microorganism species to correlate a geographic region where the production field is located with a likelihood of microorganism growth within the physical asset in view of the operating conditions.

Example 14 may include the subject matter of any of examples 11-13, wherein determining the likelihood of growth of one or more species of microorganisms comprises predicting a concentration of microorganism in parts per million (PPM).

Example 15 may include the subject matter of example 14, the operations can also include determining whether the predicted concentration of a microorganism meets or exceeds a threshold value, the threshold value establishing a minimum microorganism concentration value; and determining a mitigation strategy for the microorganism based on the predicted concentration of the microorganism meeting or exceeding the threshold value.

Example 16 may include the subject matter of example 15, the operations can also include if the predicted concentration of a microorganism meets or exceeds the threshold value: inputting the predicted concentration of the microorganism into a mechanistic model; and predicting one or both of: 1) a likelihood of corrosion in the physical asset due to the predicted concentration of the microorganism and 2) a rate of corrosion in the physical asset due to the predicted concentration of the microorganism.

Example 17 may include the subject matter of 15, the operations can also include for each of the microorganisms having a predicted concentration that meets or exceeds the threshold value, determining a metabolic pathway for each of the microorganisms; and determining a mitigation strategy for each of the microorganisms based, at least in part, on the metabolic pathway.

Example 18 may include the subject matter of any of examples 11-17, wherein the mitigation strategy comprises one or both of a) modifying the operating conditions to slow or prevent to the growth of one or more species of microorganisms; or b) adding a biocide treatment for the one or more species of microorganisms to the operating conditions.

Example 19 may include the subject matter of any of examples 11-18, the operations can also include generating a database of biomarkers for the production asset based, at least in part, on the operating conditions associated with operation of the production asset and the determined likelihood of a growth of one or more species of microorganisms in the production asset.

Example 20 is a computer-implemented system that includes one or more processors; and a non-transitory computer-readable storage medium coupled to the one or more processors and storing programming instructions for execution by the one or more processors, the programming instructions instructing the one or more processors to perform operations including inputting into a microbiologically influenced corrosion (MIC) prediction model one or more operating conditions for a physical asset in a production field; inputting into the MIC prediction model a plurality of biomarkers, each of the plurality of biomarkers being unique to a species of microorganism; determining, from the MIC prediction model, a likelihood of a growth of one or more species of microorganisms based on the operating conditions for the physical asset and the plurality of biomarkers; and determining, from the likelihood of the growth of one or more species of microorganisms, a mitigation strategy for preventing growth of the one or more species of microorganisms within the physical asset.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. For example, the signal can be a machine-generated electrical, optical, or electromagnetic signal that is generated to encode information for transmission to a suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatuses, devices, and machines for processing data, including by way of example, a programmable processor, a computer, or multiple processors or computers. The apparatus can also include special purpose logic circuitry including, for example, a central processing unit (CPU), a field-programmable gate array (FPGA), or an application-specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, such as LINUX, UNIX, WINDOWS, MAC OS, ANDROID, or IOS.

A computer program, which can also be referred to or described as a program, software, a software application, a module, a software module, a script, or code, can be written in any form of programming language. Programming languages can include, for example, compiled languages, interpreted languages, declarative languages, or procedural languages. Programs can be deployed in any form, including as stand-alone programs, modules, components, subroutines, or units for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files storing one or more modules, sub-programs, or portions of code. A computer program can be deployed for execution on one computer or on multiple computers that are located, for example, at one site or distributed across multiple sites that are interconnected by a communication network. While portions of the programs illustrated in the various figures may be shown as individual modules that implement the various features and functionality through various objects, methods, or processes, the programs can instead include a number of sub-modules, third-party services, components, and libraries. Conversely, the features and functionality of various components can be combined into single components as appropriate. Thresholds used to make computational determinations can be statically, dynamically, or both statically and dynamically determined.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computers suitable for the execution of a computer program can be based on one or more of general and special purpose microprocessors and other kinds of CPUs. The elements of a computer are a CPU for performing or executing instructions and one or more memory devices for storing instructions and data. Generally, a CPU can receive instructions and data from (and write data to) a memory.

Graphics processing units (GPUs) can also be used in combination with CPUs. The GPUs can provide specialized processing that occurs in parallel to processing performed by CPUs. The specialized processing can include artificial intelligence (AI) applications and processing, for example. GPUs can be used in GPU clusters or in multi-GPU computing.

A computer can include, or be operatively coupled to, one or more mass storage devices for storing data. In some implementations, a computer can receive data from, and transfer data to, the mass storage devices including, for example, magnetic, magneto-optical disks, or optical disks. Moreover, a computer can be embedded in another device, for example, a mobile telephone, a personal digital assistant (PDA), a mobile audio or video player, a game console, a global positioning system (GPS) receiver, or a portable storage device such as a universal serial bus (USB) flash drive.

Computer-readable media (transitory or non-transitory, as appropriate) suitable for storing computer program instructions and data can include all forms of permanent/non-permanent and volatile/non-volatile memory, media, and memory devices. Computer-readable media can include, for example, semiconductor memory devices such as random access memory (RAM), read-only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer-readable media can also include, for example, magnetic devices such as tape, cartridges, cassettes, and internal/removable disks. Computer-readable media can also include magneto-optical disks and optical memory devices and technologies including, for example, digital video disc (DVD), CD-ROM, DVD+/−R, DVD-RAM, DVD-ROM, HD-DVD, and BLU-RAY. The memory can store various objects or data, including caches, classes, frameworks, applications, modules, backup data, jobs, web pages, web page templates, data structures, database tables, repositories, and dynamic information. Types of objects and data stored in memory can include parameters, variables, algorithms, instructions, rules, constraints, and references. Additionally, the memory can include logs, policies, security or access data, and reporting files. The processor and the memory can be supplemented by, or incorporated into, special purpose logic circuitry.

Implementations of the subject matter described in the present disclosure can be implemented on a computer having a display device for providing interaction with a user, including displaying information to (and receiving input from) the user. Types of display devices can include, for example, a cathode ray tube (CRT), a liquid crystal display (LCD), a light-emitting diode (LED), and a plasma monitor. Display devices can include a keyboard and pointing devices including, for example, a mouse, a trackball, or a trackpad. User input can also be provided to the computer through the use of a touchscreen, such as a tablet computer surface with pressure sensitivity or a multi-touch screen using capacitive or electric sensing. Other kinds of devices can be used to provide for interaction with a user, including to receive user feedback including, for example, sensory feedback including visual feedback, auditory feedback, or tactile feedback. Input from the user can be received in the form of acoustic, speech, or tactile input. In addition, a computer can interact with a user by sending documents to, and receiving documents from, a device that the user uses. For example, the computer can send web pages to a web browser on a user's client device in response to requests received from the web browser.

The term “graphical user interface,” or “GUI,” can be used in the singular or the plural to describe one or more graphical user interfaces and each of the displays of a particular graphical user interface. Therefore, a GUI can represent any graphical user interface, including, but not limited to, a web browser, a touch-screen, or a command line interface (CLI) that processes information and efficiently presents the information results to the user. In general, a GUI can include a plurality of user interface (UI) elements, some or all associated with a web browser, such as interactive fields, pull-down lists, and buttons. These and other UI elements can be related to or represent the functions of the web browser.

Implementations of the subject matter described in this specification can be implemented in a computing system that includes a back-end component, for example, as a data server, or that includes a middleware component, for example, an application server. Moreover, the computing system can include a front-end component, for example, a client computer having one or both of a graphical user interface or a Web browser through which a user can interact with the computer. The components of the system can be interconnected by any form or medium of wireline or wireless digital data communication (or a combination of data communication) in a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) (for example, using 802.11 a/b/g/n or 802.20 or a combination of protocols), all or a portion of the Internet, or any other communication system or systems at one or more locations (or a combination of communication networks). The network can communicate with, for example, Internet Protocol (IP) packets, frame relay frames, asynchronous transfer mode (ATM) cells, voice, video, data, or a combination of communication types between network addresses.

The computing system can include clients and servers. A client and server can generally be remote from each other and can typically interact through a communication network. The relationship of client and server can arise by virtue of computer programs running on the respective computers and having a client-server relationship.

Cluster file systems can be any file system type accessible from multiple servers for read and update. Locking or consistency tracking may not be necessary since the locking of exchange file system can be done at application layer. Furthermore, Unicode data files can be different from non-Unicode data files.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations. It should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system including a computer memory interoperably coupled with a hardware processor configured to perform the computer-implemented method or the instructions stored on the non-transitory, computer-readable medium.

What is claimed is:

1. A computer-implemented method comprising:
inputting into a microbiologically influenced corrosion (MIC) prediction model one or more operating conditions for a physical asset in a production field;
inputting into the MIC prediction model a plurality of biomarkers, each of the plurality of biomarkers uniquely identifying one species of microorganism from a plurality of species of microorganisms;
determining, from the MIC prediction model, a likelihood of growth of one or more species of microorganisms from the plurality of species of microorganisms based on the one or more operating conditions for the physical asset and the plurality of biomarkers; and
determining, from the likelihood of the growth of one or more species of microorganisms, a mitigation strategy for preventing growth of each of the one or more species of microorganisms within the physical asset.

2. The computer-implemented method of claim 1, wherein each of the plurality of biomarkers comprises a 16S ribosomal ribonucleic acid (rRNA) gene for a corresponding microorganism species.

3. The computer-implemented method of claim 2, wherein the MIC prediction model uses a 16S rRNA gene for the corresponding microorganism species to correlate a geographic region where the production field is located with a likelihood of microorganism growth within the physical asset in view of the operating conditions.

4. The computer-implemented method of claim 1, wherein determining the likelihood of growth of one or more species of microorganisms comprises predicting a concentration of each species of microorganism in parts per million (PPM).

5. The computer-implemented method of claim 4, further comprising:
determining whether the predicted concentration of each species of microorganism meets or exceeds a threshold value, the threshold value establishing a minimum microorganism concentration value; and
determining the mitigation strategy for each microorganism based on the predicted concentration of the microorganism meeting or exceeding the threshold value.

6. The computer-implemented method of claim 5, further comprising:
if the predicted concentration of a microorganism meets or exceeds the threshold value:
inputting the predicted concentration of each of the species of microorganism into a mechanistic model; and
predicting one or both of:
1) a likelihood of corrosion in the physical asset due to the predicted concentration of each species of microorganism and
2) a rate of corrosion in the physical asset due to the predicted concentration of each species of microorganism.

7. The computer-implemented method of claim 5, further comprising:
for each of the microorganisms having a predicted concentration that meets or exceeds the threshold value,
determining a metabolic pathway for each of the species of microorganisms; and
determining the mitigation strategy for each of the species of microorganisms based, at least in part, on the metabolic pathway.

8. The computer-implemented method of claim 1, wherein the mitigation strategy comprises one or both of:
a) modifying the operating conditions to slow or prevent to the growth of one or more species of microorganisms; or
b) adding a biocide treatment for the one or more species of microorganisms to the operating conditions.

9. The computer-implemented method of claim 1, further comprising generating a database of biomarkers for the production asset based, at least in part, on the operating conditions associated with operation of the production asset and the determined likelihood of a growth of one or more species of microorganisms in the production asset.

10. The computer-implemented method of claim 1, further comprising:
testing the physical asset for MIC;
determining a concentration of one or more species of microorganisms;
determining an identity of the one or more species of microorganisms based on a genetic biomarker of the identified microorganism;
comparing the concentration of the one or more species of microorganisms with the likelihood of growth of one or more species of microorganisms predicted by the MIC prediction model; and
updating the MIC prediction model based on the comparison.

11. A non-transitory, computer-readable storage medium storing one or more instructions executable by a computer system to perform operations comprising:
inputting into a microbiologically influenced corrosion (MIC) prediction model one or more operating conditions for a physical asset in a production field;
inputting into the MIC prediction model a plurality of biomarkers, each of the plurality of biomarkers being unique to a species of microorganism;
determining, from the MIC prediction model, a likelihood of a growth of one or more species of microorganisms based on the operating conditions for the physical asset and the plurality of biomarkers; and
determining, from the likelihood of the growth of one or more species of microorganisms, a mitigation strategy for preventing growth of the one or more species of microorganisms within the physical asset.

12. The non-transitory, computer-readable storage medium of claim 11, wherein each of the plurality of biomarkers comprises a 16S ribosomal ribonucleic acid (rRNA) gene for a corresponding microorganism species.

13. The non-transitory, computer-readable storage medium of claim 12, wherein the MIC prediction model uses a 16S rRNA gene for the corresponding microorganism species to correlate a geographic region where the production field is located with a likelihood of microorganism growth within the physical asset in view of the operating conditions.

14. The non-transitory, computer-readable storage medium of claim 11, wherein determining the likelihood of growth of one or more species of microorganisms comprises predicting a concentration of microorganism in parts per million (PPM).

15. The non-transitory, computer-readable storage medium of claim 14, the operations further comprising:
determining whether the predicted concentration of a microorganism meets or exceeds a threshold value, the threshold value establishing a minimum microorganism concentration value; and
determining a mitigation strategy for the microorganism based on the predicted concentration of the microorganism meeting or exceeding the threshold value.

16. The non-transitory, computer-readable storage medium of claim 15, the operations further comprising:
if the predicted concentration of a microorganism meets or exceeds the threshold value:
inputting the predicted concentration of the microorganism into a mechanistic model; and
predicting one or both of:
1) a likelihood of corrosion in the physical asset due to the predicted concentration of the microorganism and
2) a rate of corrosion in the physical asset due to the predicted concentration of the microorganism.

17. The non-transitory, computer-readable storage medium of claim 15, the operations further comprising:
for each of the microorganisms having a predicted concentration that meets or exceeds the threshold value,
determining a metabolic pathway for each of the microorganisms; and
determining a mitigation strategy for each of the microorganisms based, at least in part, on the metabolic pathway.

18. The non-transitory, computer-readable storage medium of claim 11, wherein the mitigation strategy comprises one or both of:
a) modifying the operating conditions to slow or prevent to the growth of one or more species of microorganisms; or
b) adding a biocide treatment for the one or more species of microorganisms to the operating conditions.

19. The non-transitory, computer-readable storage medium of claim 11, the operations further comprising generating a database of biomarkers for the production asset based, at least in part, on the operating conditions associated with operation of the production asset and the determined likelihood of a growth of one or more species of microorganisms in the production asset.

20. A computer-implemented system, comprising:
one or more processors; and
a non-transitory computer-readable storage medium coupled to the one or more processors and storing programming instructions for execution by the one or more processors, the programming instructions instructing the one or more processors to perform operations comprising:
inputting into a microbiologically influenced corrosion (MIC) prediction model one or more operating conditions for a physical asset in a production field;
inputting into the MIC prediction model a plurality of biomarkers, each of the plurality of biomarkers being unique to a species of microorganism;
determining, from the MIC prediction model, a likelihood of a growth of one or more species of microorganisms based on the operating conditions for the physical asset and the plurality of biomarkers; and
determining, from the likelihood of the growth of one or more species of microorganisms, a mitigation strategy for preventing growth of the one or more species of microorganisms within the physical asset.
